(19) United States
(12) Patent Application Publication
(10) Pub. No.: US 2023/0191003 A1
(43) Pub. Date: Jun. 22, 2023

(54) COMPOSITE SCAFFOLD MATERIAL, PREPARATION METHOD THEREFOR AND USE THEREOF

(71) Applicant: SUZHOU BOCHUANG TONGKANG PHARMACEUTICAL TECHNOLOGY CO. , LTD., Kunshan, Jiangsu (CN)

(72) Inventors: Guangwan LIU, Suzhou,Jiangsu (CN); Yinghua SHAO, Suzhou,Jiangsu (CN); Fang ZHAO, Suzhou,Jiangsu (CN); Hongyuan QU, Suzhou,Jiangsu (CN); Deli SONG, Suzhou,Jiangsu (CN)

(73) Assignee: Suzhou Novovita Bio-products Co., Ltd., Suzhou,Jiangsu (CN)

(21) Appl. No.: 16/964,198

(22) PCT Filed: Dec. 25, 2018
(86) PCT No.: PCT/CN2018/123280

§ 371 Date: Oct. 28, 2020

(30) Foreign Application Priority Data
Jan. 23, 2018 (CN) 201810063541.7

(51) Int. Cl. A61L 27/44; A61L 27/54

(52) U.S. Cl. CPC A61L 27/44; A61L 27/54; D04H 1/728

(57) ABSTRACT

The present disclosure relates to the technical field of medicines, and in particular, to a composite scaffold material, a preparation method therefor and use thereof. The present disclosure provides a composite scaffold material, including a charged fiber framework material. The fiber framework material is coated with positively charged biocompatible materials and negatively charged biocompatible materials alternately, by means of electrostatic attraction. The composite scaffold material of the present disclosure overcomes the defects of traditional scaffold materials, such as poor hydrophilicity, biocompatibility, histiocyte adhesion ability, and biological induction activity. The composite scaffold material has better applicability when used for tissue adhesion, closure, leaking stoppage, hemostasis, isolation, repair, and adhesion prevention, and can also be used for preparing a drug carrier (e.g., sustained release carrier) and a tissue engineering scaffold material. Therefore the composite scaffold material has a wide range of industrial uses.

### CROSS REFERENCES TO RELATED APPLICATIONS

This is a Sect. 371 National Stage application of a PCT International Application No. PCT/CN2018/123280, filed on Dec. 25, 2018, which claims the benefits of priority to Chinese Patent Application No. 2018100635417, entitled “Composite Scaffold Material, Preparation Method Therefor and Use Thereof”, filed with CNIPA on Jan. 23, 2018, the contents of which are incorporated herein by reference in its entirety.

### TECHNICAL FIELD

The present disclosure relates to the technical field of medicines, and in particular, to a composite scaffold material, a preparation method therefor and use thereof.

### BACKGROUND

Common in-vivo implanted absorbable scaffold materials, such as degradable polymer electrospun scaffold made of poly(lactic-co-glycolic acid) (PLGA), polylactic acid (PLA) or polycaprolactone (PCL), have poor hydrophilicity, biocompatibility, histiocyte adhesion ability and biological induction activity. Therefore, these scaffold materials cannot induce and promote the regeneration and repair of defective tissues well. In addition, the large volume shrinkage of these scaffolds in body fluids would further affect the use effect. The use of acellular matrix material would cause immune rejection response due to the incomplete removal of immunogenicity. Biopolymer material, such as collagen sponge, has good biocompatibility, but the poor mechanical properties such as mechanical strength, as well as the easy-to-collapse structure, make it quickly degrade after implantation, and the tissue is not completely repaired when the material is fully degraded. The polymer casting film has a dense surface, which makes it difficult for the host cells to migrate into the material and only attach to the surface of the material, leading to a decline in the repair effect. Most tissue repair regenerative scaffolds are prone to fracture and delamination, resulting in separation during use or volume shrinkage after implantation in the body, affecting the treatment effect. Most tissue repair patches are cross-linked with cross-linking agents, which can improve mechanical properties after cross-linking and thus prolong the degradation time. However, excessive use of cross-linking agents not only has certain tissue toxicity, but also tends to lead to calcification.

### SUMMARY

The present disclosure provides a composite scaffold material, a preparation method therefor and use thereof.

A first aspect of the present disclosure provides a composite scaffold material, including a charged fiber framework material. The fiber framework material is coated with positively charged biocompatible materials and negatively charged biocompatible materials alternately by means of electrostatic attraction

In the composite scaffold material of the present disclosure, the fiber framework material generally includes fibers. For example, the fiber constituting the fiber framework material may be formed by electrospinning and/or 3D printing, and may generally be distributed in the composite scaffold material evenly. Those skilled in the art may adjust the shape of the fiber framework material and the shape of the composite scaffold material according to needs. For example, the fiber framework material may be formed by stacked fibers. For another example, the fiber framework material and/or the composite scaffold material can usually be various regular or irregular shapes such as layers, spheres, cylinders (hollow or non-hollow). The formed structure may be various two-dimensional or three-dimensional regular or irregular shapes such as membranes, tubes, spheres. In a specific embodiment of the present disclosure, the fiber framework material may be a flat layer (for example, a membrane, etc.). For another example, the diameter (thickness) of the fibers constituting the fiber framework material may be 3 nm-20000 nm, 3 nm-10 nm, 10 nm-20 nm, 20 nm-30 nm, 30 nm-50 nm, 50 nm-70 nm, 70 nm-100 nm, 100 nm-150 nm, 150 nm-200 nm, 200 nm-250 nm, 250 nm-300 nm, 300 nm-350 nm, 350 nm-400 nm, 400 nm-450 nm, 450 nm-500 nm, 500 nm-1000 nm, 1000 nm-5000 nm, 5000 nm-10000 nm, 10000 nm-20000 nm, 20000 nm-30000 nm, 30000 nm-40000 nm, 40000 nm-50000 nm, 50000 nm-60000 nm. For another example, the volume percentage of fibers in the fiber framework material may be 20%-90%, 20%-30%, 30%-40%, 40%-50%, 50%-60%, 60%-70%, 70%-80% or 80%-90%.

In the composite scaffold material of the present disclosure, the fibers constituting the fiber framework material are generally charged (positive and/or negative). The fiber may be a fiber with an electric charge of its own, or may be a fiber with an electric charge after charge modification treatment, so that the positively charged biocompatible materials and negatively charged biocompatible materials can be coated on the fiber framework material alternately by electrostatic attraction. The charge modification treatment generally refers to a treatment method in which the fibers are modified by a certain physical method and/or chemical method (for example, adhering or adding charged substances to the fibers, or modifying or plasma modifying the fiber surface), so that the resulting fibers are positively charged and/or a negatively charged. Those skilled in the art may select an appropriate modification method to charge-modify the fibers according to the type of the fibers and the type of the carried charge after modification. These methods should be known to those skilled in the art. For example, the material obtained by the charge modification treatment may carry a positively charged group or a negatively charged group, so that the fibers constituting the fiber framework material are charged. The positively charged group may include but is not limited to any one of an amino group, a quaternary ammonium group, or combinations thereof. The negatively charged group may include but is not limited to any one of a mercapto group, a carboxyl group, a sulfonic group, a phosphate group, or a combination thereof.

In the composite scaffold material of the present disclosure, the fibers constituting the fiber framework material are generally biocompatible materials. The biocompatibility generally refers to good compatibility between the material (for example, an inactive material) and the host. For example, the biocompatibility of fiber framework materials usually refers to good affinity, low immune response, and no cytotoxicity after implanting in vivo. Specifically, the materials comply with the relevant provisions of the International Standards Organization (ISO) 10993 and/or National Standard GB/T16886. The fibers constituting the fiber framework material may be a polymer material, which generally refers to a material composed of a polymer compound (generally referring to a compound with a relative molecular mass (for example, weight-average molecular weight) ≥1000, ≥1500, or ≥2000) as a matrix. The polymer material may be a natural biopolymer material and/or an organic synthesized polymer material. The natural polymer material generally refers to a polymer substance existing in animals, plants or other living organisms. The natural polymer material may specifically include but is not limited to any one of polylysine, polyglutamic acid, collagen, silk fibroin, soy protein, elastin, hyaluronic acid, chitosan, carboxymethyl chitosan, carboxymethyl dextran, carboxymethyl glucose, heparin, alginic acid, chondroitin sulfate, carboxymethyl starch, carboxymethyl cellulose, other modified materials, or combinations thereof. The organic synthesized polymer material usually refers to a polymer material prepared by a chemical synthesis method, and may specifically include but is not limited to any one of polylactic acid, polyglycolic acid, polycaprolactone, polylactic acid glycolic acid copolymer, polylactic acid caprolactone copolymer, polydioxanone, trimethylene carbonate, polyethylene, polypropylene, polytetrafluoroethylene, polyurethane, polyethylene glycol, polyvinylpyrrolidone, or combinations thereof.

In the composite scaffold material of the present disclosure, the fibers are doped with charged doping materials. The doped fibers have a stronger charge after doping with charged doping materials. The doping material may be a natural biopolymer material, and may specifically be a natural biopolymer material as described above. A person skilled in the art may select a suitable doping ratio according to needs. For example, the doping material may be mixed with the fibers in any ratio. In specific, the doping material may account for no more than 1 wt %, 1-5 wt %, 5-10 wt %, 10-20 wt %, 20-30 wt %, 30-40 wt %, 40-50 wt %, 50-60 wt %, 60-70 wt %, 70-80 wt %, 80-90 wt % or higher of the total fiber mass after doping.

In the composite scaffold material of the present disclosure, the fiber framework material may be supplemented with functional factors and/or functional polypeptides. The functional factor generally refers to a substance that can regulate the functions of the human body by activating enzyme activity or other pathways. For example, the functional factor in the fiber framework material may include but not limited to fibronectin, laminin, vascular endothelial growth factor, fibrinogen, epidermal growth factor, fibroblast growth factor, transforming growth factor, bone morphogenetic protein, insulin-like growth factor, platelet-derived growth factor, hydroxyapatite, strontium chloride, thrombin, or combinations thereof. The amount of the added functional factor in the fiber framework material may be not more than 10 wt %, not more than 20 wt %, not more than 30 wt %, not more than 40 wt %, not more than 50 wt %, not more than 1 wt %, 1-2 wt %, 2-4 wt %, 4-6 wt %, 6-8 wt %, 8-10 wt %, 10-15 wt %, 15-20 wt %, 20-25 wt %, 25-30 wt %, 30-35 wt %, 35-40 wt %, 40-45 wt % or 45-50 wt %. The functional polypeptide generally refers to a class of functional factor that can be a polypeptide. For example, the functional polypeptide in the fiber framework material may include but is not limited to any one of RGD polypeptide, polypeptide containing -arginine-glycine-aspartic acid, polypeptide containing -valine-glycine-valine-alanine-proline-glycine, polypeptide containing -isoleucine-lysine-valine-alanine-valine, or combinations thereof. The amount of the added functional polypeptide in the fiber framework material may be not more than 10 wt %, not more than 20 wt %, not more than 30 wt %, not more than 40 wt %, not more than 50 wt %, not more than 1 wt %, 1-2 wt %, 2-4 wt %, 4-6 wt %, 6-8 wt %, 8-10 wt %, 10-15 wt %, 15-20 wt %, 20-25 wt %, 25-30 wt %, 30-35 wt %, 35-40 wt %, 40-45 wt % or 45-50 wt %.

In the composite scaffold material of the present disclosure, coating the framework material with positively charged biocompatible materials and negatively charged biocompatible materials alternately by means of electrostatic attraction may be realized by an electrostatic self-assembly method. The electrostatic self-assembly method should be known to those skilled in the art. For example, a positively-charged biocompatible material and/or a negatively-charged biocompatible material may be dissolved in an appropriate amount of solvent, and the self-assembly is achieved by spraying and/or soaking.

In the composite scaffold material of the present disclosure, the positively charged biocompatible materials and/or negatively charged biocompatible materials are generally biocompatible materials. The biocompatibility of the positively charged biocompatible materials and/or negatively charged biocompatible materials generally refers to good affinity, low immune response, and no cytotoxicity after implanting in vivo. Specifically, the materials comply with the relevant provisions of the International Standards Organization (ISO) 10993 and/or National Standard GB/T16886. The positively charged biocompatible material may be a positively charged material, and the positively charged material generally refers to a material with a positive charge, which may specifically include but is not limited to any one of polylysine, collagen, silk fibroin, fibronectin, laminin, fibrinogen, chitosan, or combinations thereof. The negatively charged biocompatible material may be a negatively charged material, the negatively charged material usually refers to a material with a negative charge, which may specifically include but is not limited to any one of polyglutamic acid, collagen, silk fibroin, fibronectin, laminin, fibrinogen (protein is an ampholyte, which can be charged differently by adjusting the pH of the solution), hyaluronic acid, carboxymethyl chitosan, carboxymethyl dextran, carboxymethyl glucose, heparin, alginic acid, chondroitin sulfate, carboxymethyl starch, carboxymethyl cellulose, or combinations thereof.

In the composite scaffold material of the present disclosure, the positively charged biocompatible materials and/or negatively charged biocompatible materials may be supplemented with functional factors and/or functional polypeptides. For example, the functional factor in the positively charged biocompatible materials and/or negatively charged biocompatible materials may include but not limited to fibronectin, laminin, vascular endothelial growth factor, fibrinogen, nerve growth factor, epidermal growth factor, fibroblast growth factor, transforming growth factor, bone morphogenetic protein, insulin-like growth factor, platelet-derived growth factor, hydroxyapatite, strontium chloride, thrombin, or combinations thereof. The amount of the added functional factor in the positively charged biocompatible materials and/or negatively charged biocompatible materials may be not more than 10 wt %, not more than 20 wt %, not more than 30 wt %, not more than 40 wt %, not more than 50 wt %, not more than 1 wt %, 1-2 wt %, 2-4 wt %, 4-6 wt %, 6-8 wt %, 8-10 wt %, 10-15 wt %, 15-20 wt %, 20-25 wt %, 25-30 wt %, 30-35 wt %, 35-40 wt %, 40-45 wt % or 45-50 wt %. For example, the functional polypeptide in the positively charged biocompatible materials and/or negatively charged biocompatible materials may include but are not limited to any one of RGD polypeptide, polypeptide containing -arginine-glycine-aspartic acid, polypeptide containing -valine-glycine-valine-alanine-proline-glycine, polypeptide containing -isoleucine-lysine-valine-alanine-valine, or combinations thereof. The amount of the added functional polypeptide in the positively charged biocompatible materials and/or negatively charged biocompatible materials may be not more than 10 wt %, not more than 20 wt %, not more than 30 wt %, not more than 40 wt %, not more than 50 wt %, not more than 1 wt %, 1-2 wt %, 2-4 wt %, 4-6 wt %, 6-8 wt %, 8-10 wt %, 10-15 wt %, 15-20 wt %, 20-25 wt %, 25-30 wt %, 30-35 wt %, 35-40 wt %, 40-45 wt % or 45-50 wt %.

In the composite scaffold material of the present disclosure, the positively charged biocompatible material layers and negatively charged biocompatible material layers are coated with the positively charged biocompatible materials and negatively charged biocompatible materials alternately. Those skilled in the art may adjust parameters such as the thickness and layer number of the positively charged biocompatible material layers and/or the negatively charged biocompatible material layers according to needs. For example, the thickness of each layer of the positively charged biocompatible material layers and/or the negatively charged biocompatible material layers may be 0.1-100 micrometers, 0.1-1 micrometers, 1-5 micrometers, 5-10 micrometers, 10-20 micrometers, 20-40 micrometers, 40-60 micrometers, 60-80 micrometers or 80-100 micrometers. The total layers of the positively charged biocompatible material layers and the negatively charged biocompatible material layers which are alternately superimposed may be 2-200 layers, 2-10 layers, 10-30 layers, 30-50 layers, 50-100 layers, 100-150 layers or 150-200 layers.

In the composite scaffold material of the present disclosure, a person skilled in the art can appropriately adjust the proportions of the fiber framework material, the positively charged biocompatible material, and the negatively charged biocompatible material according to needs. For example, the fiber framework material may account for 5-95%, 5-15%, 15-25%, 25-35%, 35-45%, 45-55%, 55-65%, 65-75%, 75-85% or 85-95% of the total mass of the composite scaffold material. For another example, the positively charged biocompatible material may account for 5-95%, 5-15%, 15-25%, 25-35%, 35-45%, 45-55%, 55-65%, 65-75%, 75-85% or 85-95% of the total mass of the composite scaffold material. For another example, the negatively charged biocompatible material may account for 5-95%, 5-15%, 15-25%, 25-35%, 35-45%, 45-55%, 55-65%, 65-75%, 75-85% or 85-95% of the total mass of the composite scaffold material. For another example, The positively charged biocompatible material and negatively charged biocompatible material account for 5-95%, 5-15%, 15-25%, 25-35%, 35-45%, 45-55%, 55-65%, 65-75%, 75-85% or 85-95% of the total mass of the composite scaffold material.

A second aspect of the present disclosure provides a method for preparing the composite scaffold material, including:

The fiber framework materials are formed with polymer biocompatible materials by electrospinning and/or 3D printing method, then the fiber framework materials are coated with positively charged biocompatible materials and negatively charged biocompatible materials alternately by electrostatic self-assembly.

In the preparation method of the composite scaffold material of the present disclosure, a person skilled in the art may select a suitable process condition to prepare the fiber framework materials according to the type and parameters of the required fibers. For example, electrospinning and/or 3D printing may usually be carried out in the presence of a solvent, and the polymer biocompatible material may be dissolved in a suitable solvent to perform electrospinning and/or 3D printing. Solvents used in electrospinning and/or 3D printing include but are not limited to any one of formic acid, acetic acid, ethanol, acetone, dimethylformamide, dimethylacetamide, tetrahydrofuran, dimethyl sulfoxide, hexafluoroisopropanol, trifluoroethanol, dichloromethane, trichloromethane, methanol, chloroform, dioxane, trifluoroethane, trifluoroacetic acid, water, normal saline, various buffer solutions, or combinations thereof. The injection speed in electrospinning may be 0.001-90 mm/min, 0.001-0.01 mm/min, 0.01-0.1 mm/min, 0.1-1 mm/min, 1-5 mm/min, 5-10 mm/min, 10-20 mm/min, 20-30 mm/min, 30-40 mm/min, 40-50 mm/min, 50-60 mm/min, 60-70 mm/min, 70-80 mm/min or 80-90 mm/min. The positive voltage of the high voltage generator in electrospinning may be 0.1-40 kv, 0.1-0.5 kv, 0.5-1 kv, 1-5 kv, 5-10 kv, 10-15 kv, 15-20 kv, 20-25 kv, 25-30 kv, 30-35 kv or 35-40 kv. For another example, the negative voltage of the high voltage generator in electrospinning may be 0.1-10 kV, 0.1-0.5 kV, 0.5-1 kV, 1-3 kV, 3-5 kV, or 5-10 kV. The distance between the spinning injector and the receiving device in electrospinning may be 3-30 cm, 3-5 cm, 5-10 cm, 10-15 cm, 15-20 cm, 20-25 cm, or 25-30 cm. The 3D printing speed may be 0.001-100 mm/min, 0.001-0.01 mm/min, 0.01-0.1 mm/min, 0.1-1 mm/min, 1-5 mm/min, 5-10 mm/min, 10-20 mm/min, 20-30 mm/min, 30-40 mm/min, 40-50 mm/min, 50-60 mm/min, 60-70 mm/min, 70-80 mm/min or 80-100 mm/min. The 3D printing temperature (For example, organic polymer materials can be 3D-printed in a solvent or molten state) may be −20-400° C., −20-10° C., −10-0° C., 0-10° C., 10-20° C., 20-30° C., 30-40° C., 40-50° C., 50-60° C., 60-70° C., 70-80° C., 80-90° C., 90-100° C., 100-200° C., 200-300° C. or 300-400° C.

In the preparation method of the composite scaffold material of the present disclosure, a person skilled in the art may select a suitable process condition to coat the fiber framework materials with positively charged biocompatible materials and negatively charged biocompatible materials alternately by electrostatic self-assembly according to the type and parameters of the required fibers. For example, in order to coat the material with positively charged biocompatible materials and negatively charged biocompatible materials alternately, electrostatic self-assembly may be achieved by spraying and/or soaking, and further by drying and sterilizing. Electrostatic self-assembly is carried out in the presence of a solvent. Positively charged biocompatible materials and/or negatively charged biocompatible materials can be dissolved in an appropriate amount of solvent to perform electrostatic self-assembly. The solvents used in electrostatic self-assembly include but are not limited to any one of formic acid solution, acetic acid solution, hydrochloric acid solution, phosphoric acid solution, sulfuric acid solution, water, alkaline solution, physiological saline, various saline solutions and buffer solutions, or combinations thereof. A person skilled in the art may select a suitable method for drying and/or sterilizing during the preparation process. Specific drying methods may include, but are not limited to, freeze drying, drying, vacuum drying, or natural drying. Methods include but are not limited to irradiation sterilization and/or ethylene oxide chemical sterilization. Specific sterilizing methods may include, but are not limited to, radiation sterilization and/or ethylene oxide chemical sterilization.

A third aspect of the present disclosure provides the use of the composite scaffold material in the preparation of biocompatible materials.

The composite scaffold material of the present disclosure has good biocompatibility, and can be used to prepare carrier materials, tissue engineering scaffold materials and the like. The carrier material generally refers to a carrier that can be used to carry other substances, specifically including but not limited to a drug carrier materials, etc. The drug carrier material can generally be used to carry various drugs. The tissue engineering scaffold material usually refers to a material that can be implanted into vivo, combine with the living tissue cells, and perform certain functions in the specific replaced tissue. The tissue engineering scaffold materials may include but are not limited to medical materials such as artificial meninges, artificial blood vessels, nerve conduits, bone repair scaffold, artificial skin and patches.

The composite scaffold material of the present disclosure, coating the fiber framework material with positively charged biocompatible materials and negatively charged biocompatible materials alternately by means of electrostatic attraction, overcomes the defects of traditional scaffold materials, such as poor hydrophilicity, biocompatibility, histiocyte adhesion ability and biological induction activity. The composite scaffold material has better applicability when used for tissue adhesion, closure, leaking stoppage, hemostasis, isolation, repair, and adhesion prevention, and can also be used for preparing a drug carrier (e.g., sustained release carrier) and a tissue engineering scaffold material. Therefore, the composite scaffold material has a wide range of industrial uses. The composite scaffold material of the present disclosure simulates the extracellular matrix through the combination of organic synthesized polymer material and biopolymer material, providing living space for the cells to anchor, grow and reproduce, and to obtain nutrients and performs metabolism. The in-vivo degradation rate of the materials can be regulated by the optimal combination of the materials. The addition of functional factors and functional structural polypeptides in the composite scaffold material helps the adhesion and expansion of cells, and realizes the multifunctional use of the material in the repair and regeneration of damaged tissues. Therefore, the composite scaffold material has high biological induction activity. The nano-scale material obtained by the method of alternately superimposing polyelectrolytes makes the physicochemical properties of the product conducive to cell adhesion and gene expression. In particular, the mechanical properties of the material are strengthened, which makes the material less likely to deform and shrink. Thus, the toxic and side effects caused by excessive use of cross-linking agents are avoided. Moreover, products for different needs can be produced according to different parts used, and the preparation and operation are convenient. To sum up, the composite scaffold material has a good industrialization prospect.

### BRIEF DESCRIPTION OF THE DRAWINGS

FIG. 1 is a schematic diagram of the scanning electron microscope test results of Embodiment 10 of the present disclosure.

### DETAILED DESCRIPTION OF THE PREFERRED EMBODIMENTS

The embodiments of the present disclosure will be described below through exemplary embodiments. Those skilled in the art can easily understand other advantages and effects of the present disclosure according to contents disclosed by the specification. The present disclosure can also be implemented or applied through other different exemplary embodiments. Various modifications or changes can also be made to all details in the specification based on different points of view and applications without departing from the spirit of the present disclosure.

It should be noted that processing equipment or devices not specifically noted in the following embodiments are all conventional equipment or devices in the field.

In addition, it should be understood that one or more method steps mentioned in the present disclosure are not exclusive of other method steps that may exist before or after the combined steps or that other method steps may be inserted between these explicitly mentioned steps, unless otherwise stated; it should also be understood that the combined connection relationship between one or more equipment/devices mentioned in the present disclosure does not exclude that there may be other equipment/devices before or after the combined equipment/devices or that other equipment/devices may be inserted between these explicitly mentioned equipment/devices, unless otherwise stated. Moreover, unless otherwise stated, the numbering of each method step is only a convenient tool for identifying each method step, and is not intended to limit the order of each method step or to limit the scope of the present disclosure. The change or adjustment of the relative relationship shall also be regarded as the scope in which the present disclosure may be implemented without substantially changing the technical content.

### Embodiment 1

Preparing a drying culture dish (ϕ=12 cm, washed, sterilized and dehydrogenated), using as an electrospinning receiver; weighing 1 g PLGA and 0.2 g collagen, adding 10 ml hexafluoroisopropanol to prepare an electrospinning A solution of degradable polymer material PLGA and cationic group material; adding the above solution to the injector of the electrospinning device, adjusting the injection rate of the micro-injection pump to 0.1-0.3 mm/min, adjusting the positive voltage to 15V and negative voltage to 2V, adjusting the receiving distance of the receiving device to 15 cm; the fibers are received as a film-like structure to prepare an electrospun membrane A.

Weighing 0.5 g carboxymethyl dextran, dissolving in water and adjusting pH=6 to prepare B solution with anionic groups; pouring the above solution into the culture dish that has prepared electrospun membrane A, soaking, drying and forming membrane, and assembling a layer of carboxymethyl dextran membrane B on electrospun membrane A.

Continuing to prepare an electrospun membrane A on the carboxymethyl dextran membrane, assembling a layer of carboxymethyl dextran membrane B on the electrospun membrane A, and coating 20 times alternately, rinsing with water-for-injection, freeze-drying, peeling off the membrane, and sterilizing the product by irradiation.

### Embodiment 2

Preparing a drying culture dish (,washed, sterilized and dehydrogenated) using as an electrospinning receiver; weighing 1 g PLA and 0.2 g chitosan and adding 10 ml hexafluoroisopropanol to prepare an A solution of degradable polymer material PLA and cationic group material; adding the above solution to the injector of the electrospinning device, adjusting the injection rate of the micro-injection pump to 0.5 mm/min, adjusting the positive voltage to 15V and negative voltage to 2V, adjusting the receiving distance of the receiving device to 10 cm, and electrospinning 20 layers of electrospun membrane A.

Weighing 0.5 g carboxymethyl dextran, dissolving in water and adjusting pH=6 to prepare B solution with anionic group material. Pouring the above solution into the polypropylene culture dish that has prepared electrospun membrane A, soaking, drying and forming membrane. Sterilizing the product with ethylene oxide.

### Embodiment 3

Preparing a drying culture dish (4)=12 cm, washed, sterilized and dehydrogenated),; weighing 0.5 g hyaluronic acid, dissolving in water and adjusting pH=6 to prepare A solution with anionic group material. Spraying the above A solution on the culture dish and drying and forming membrane A; weighing 0.5 g chitosan, dissolving in water and adjusting pH=4 to prepare B solution of cationic group material; spraying B solution on the membrane A to make the B solution to assemble under the action of electrostatic force; coating 20 times alternately by electrostatic assembly to obtain the electrostatic assembly membrane. Weighing 1 g PCL and 0.2 g polylysine and adding 10 ml hexafluoroisopropanol to prepare an electrospinning C solution of degradable polymer material PCL and cationic group material; adding the above solution to the injector of the electrospinning device, adjusting the injection rate of the micro-injection pump to 1.0 mm/min, adjusting the positive voltage to 15V and negative voltage to 3V, adjusting the receiving distance of the receiving device to 20 cm, and wrapping the electrospinning fibers on the prepared electrostatic assembly membrane, and freeze-drying. Sterilizing the product by irradiation.

### Embodiment 4

Preparing a drying polypropylene culture dish (ϕ=12 cm, washed, sterilized and depyrogenated), and using as an electrospinning receiver; weighing 1 g PLGA and 0.2 g hyaluronic acid and adding 10 ml hexafluoroisopropanol to prepare an electrospinning A solution of degradable polymer material PLGA and anionic group material; adding the above solution to the injector of the electrospinning device, adjusting the injection rate of the micro-injection pump to 0.3 mm/min, adjusting the positive voltage to 15V and negative voltage to 2V, adjusting the receiving distance of the receiving device to 15 cm; the fibers are received as a film-like structure to prepare an electrospun membrane A.

Weighing 0.5 g collagen, dissolving in water and adjusting pH=6 to prepare B solution with cationic groups; pouring the above solution into the culture dish that has prepared electrospun membrane A, soaking, drying and forming membrane, and assembling a layer of collagen membrane B on the electrospun membrane A.

Continuing to prepare an electrospun membrane A on the collagen membrane, assembling a layer of collagen membrane B on the electrospun membrane A, and repeating the superimposition 50 times, rinsing with water-for-injection, freeze-drying, peeling off the membrane, and sterilizing the product by irradiation.

### Embodiment 5

Preparing a culture dish (washed, sterilized and dehydrogenated) with a diameter of 12 cm, drying, and using as an electrospinning receiver; weighing 1 g PLA and 0.2 g carboxymethyl dextran and adding 10 ml hexafluoroisopropanol to prepare an A solution of degradable polymer material PLA and anionic group material; adding the above solution to the injector of the electrospinning device, adjusting the injection rate of the micro-injection pump to 0.1-0.3 mm/min, adjusting the positive voltage to 15V and negative voltage to 2V, adjusting the receiving distance of the receiving device to 15 cm, and electrospinning 50 layers of electrospun membrane A.

Weighing 0.5 g chitosan, dissolving in water and adjusting pH=4 to prepare B solution with cationic group material. Pouring the above solution into the culture dish that has prepared electrospun membrane A, soaking and drying into a membrane. Sterilizing the product with ethylene oxide.

### Embodiment 6

Preparing a culture dish (washed, sterilized and dehydrogenated) with a diameter of 12 cm, drying, and using as an electrospinning receiver; weighing 1 g PLA and 0.2 g hyaluronic acid and adding 10 ml hexafluoroisopropanol to prepare an A solution of degradable polymer material PLA and anionic group material; adding the above solution to the injector of the electrospinning device, adjusting the injection rate of the micro-injection pump to 2.5 mm/min, adjusting the positive voltage to 20V and negative voltage to 4V, adjusting the receiving distance of the receiving device to 25 cm, and electrospinning 20 layers of electrospun membrane A.

Weighing 0.5 g collagen, dissolving in acetic acid solution and adjusting pH=6 to prepare B solution with cationic group material; adding 10% thrombin (concentration: 2000 IU/ml) to B solution. Pouring the above solution into the culture dish that has prepared electrospun membrane A, soaking and drying into a membrane. Sterilizing the product by irradiation.

### Embodiment 7

Preparing a culture dish (washed, sterilized and dehydrogenated) with a diameter of 12 cm, drying, and using as an electrospinning receiver; weighing 1 g PLGA and 0.2 g collagen and adding 10 ml hexafluoroisopropanol to prepare an electrospinning A solution of degradable polymer material PLGA and cationic group material, adding RGD polypeptide to A solution; adding the above solution to the injector of the electrospinning device, adjusting the injection rate of the micro-injection pump to 0.8 mm/min, adjusting the positive voltage to 12V and negative voltage to 2V, adjusting the receiving distance of the receiving device to 10 cm; the fibers are received as a film-like structure to prepare an electrospun membrane A.

Weighing 0.5 g carboxymethyl dextran, dissolving in water and adjusting pH=6 to prepare B solution with anionic groups; pouring the above solution into the culture dish that has prepared electrospun membrane A, soaking and drying into a membrane, and assembling a layer of carboxymethyl dextran membrane B on electrospun membrane A.

Continuing to prepare an electrospun membrane A on the carboxymethyl dextran membrane, assembling a layer of carboxymethyl dextran membrane B on the electrospun membrane A, and repeating the superimposition 20 times. Rinsing with water-for-injection, freeze-drying, and sterilizing the product with ethylene oxide.

### Embodiment 8

Using a rotatable hollow duct (washed, sterilized and dehydrogenated) as an electrospinning receiver; weighing 1 g PLGA and 0.2 g collagen and adding 10 ml hexafluoroisopropanol to prepare an electrospinning A solution of degradable polymer material PLGA and cationic group material; adding the above solution to the injector of the electrospinning device, adjusting the injection rate of the micro-injection pump to 0.2 mm/min, adjusting the positive voltage to 10V and negative voltage to 1V, adjusting the receiving distance of the receiving device to 15 cm, and preparing a tubular electrospun membrane.

Weighing 0.5 g hyaluronic acid, dissolving in water and adjusting pH=6 to prepare B solution with anionic groups; soaking the above electrospinning duct into the B solution for electrostatic assembly, drying into a membrane, rinsing with water-for-injection, freeze-drying, and sterilizing the product by irradiation.

### Embodiment 9

Using a roller as an electrospinning receiver, and wrapping a layer of aluminum foil (washed, sterilized and dehydrogenated) on the roller; weighing 1 g PLGA and 0.2 g collagen and adding 10 ml hexafluoroisopropanol to prepare an electrospinning A solution of degradable polymer material PLGA and cationic group material; adding the above solution to the injector of the electrospinning device, adjusting the injection rate of the micro-injection pump to 0.4 mm/min, adjusting the positive voltage to 15V and negative voltage to 2V, adjusting the receiving distance of the receiving device to 15 cm; the fibers are received as a film-like structure, which is torn off from the aluminum foil to prepare an electrospun membrane A.

Weighing 0.5 g sodium carboxymethylcellulose, dissolving in water and adjusting pH=6 to prepare B solution with anionic groups, adding 0.5 g metformin hydrochloride to B solution to dissolve; soaking the above electrospun membrane into the B solution for electrostatic assembly, drying into a membrane, rinsing with water-for-injection, freeze-drying, and sterilizing the product by irradiation.

### Embodiment 10

Preparing a culture dish (washed, sterilized and dehydrogenated) with a diameter of 12 cm, drying, and using as an electrospinning receiver; weighing 1 g PLGA, adding 10 ml acetone to dissolve, and preparing an electrospinning A solution of degradable polymer material PLGA; adding the above solution to the injector of the electrospinning device, adjusting the injection rate of the micro-injection pump to 0.1-0.3 mm/min, adjusting the positive voltage to 15V and negative voltage to 2V, adjusting the receiving distance of the receiving device to 15 cm; the fibers are received as a film-like structure to prepare an electrospun membrane A.

Weighing 0.5 g carboxymethyl dextran, dissolving in water and adjusting pH=6 to prepare B solution with anionic groups; pouring the above solution into the culture dish that has prepared electrospun membrane A, soaking and drying into a membrane, and assembling a layer of carboxymethyl dextran membrane B on electrospun membrane A.

Weighing 0.5 g collagen, dissolving in acetic acid solution to dissolve, and preparing C solution with cationic groups; pouring the above solution into the culture dish, soaking and drying into a membrane, and assembling a layer of collagen on the membrane B through electrostatic assembly. Alternately superimposing B solution and C solution for 20 times and then superimposing A by electrospinning; continuously and alternately repeating 5 times, rinsing with water-for-injection, freeze-drying, and sterilizing the product with ethylene oxide.

### Embodiment 11

Weighing 1 g PLGA, adding 10 ml acetone to dissolve, preparing 3D printing A solution of degradable polymer material PLGA; adding the above solution to the 3D printing device, importing the meningeal tissue related data into the computer, 3D printing into A membrane, the printing rate is 0.1 mm/min, the printing temperature is 10° C.

Weighing 0.1 g hyaluronic acid, dissolving in water and adjusting pH=6 to prepare B solution with anionic groups; placing the above 3D printing membrane A into the solution for soaking, drying into a membrane, and assembling a layer of carboxymethyl dextran membrane B on the 3D printing membrane A.

Weighing 0.1 g collagen, dissolving in acetic acid solution to dissolve, and preparing C solution with cationic groups; placing the above 3D printing membrane into the solution C for soaking, drying into a membrane, and assembling a layer of collagen on the membrane B through electrostatic assembly. Alternately superimposing B solution and C solution for 20 times; rinsing with water-for-injection, freeze-drying, and sterilizing the product by irradiation.

### Embodiment 12

The samples prepared by the Embodiments were tested for their hydrophilicity, mechanical properties, immersion shrinkage, and scanning electron microscope. For the preparation method of the PLGA electrospun membrane used in this Embodiment, please refer to the preparation method of the electrospun membrane A in Embodiment 10.

1. Hydrophilicity. The hydrophilicity of the samples was evaluated by the contact angle, and the technical standard for measuring the contact angle refers to GB/T 30447-2013. The results are shown in Table 1:

TABLE 1

| Samples | Contact angle° |
|---|---|
| PLGA electrospun membrane | 140.3 |
| composite scaffold material of Embodiment 1 | 66.3 |
| composite scaffold material of Embodiment 2 | 70.1 |
| composite scaffold material of Embodiment 3 | 85.2 |
| composite scaffold material of Embodiment 4 | 77.9 |
| composite scaffold material of Embodiment 5 | 65.9 |
| composite scaffold material of Embodiment 6 | 59.4 |
| composite scaffold material of Embodiment 7 | 71.2 |
| composite scaffold material of Embodiment 8 | 62.4 |
| composite scaffold material of Embodiment 9 | 64.6 |
| composite scaffold material of Embodiment 10 | 66.6 |
| composite scaffold material of Embodiment 11 | 73.3 |

The test results show that all the composite scaffold materials made by the Embodiments of this technique have a contact angle of less than 90°. The single PLGA electrospun membrane has a contact angle of 140°, indicating that the scaffold material has changed from highly hydrophobic to hydrophilic.

2. Mechanical property. The technical standard for measuring mechanical properties refers to GB/T 1014.1-2006. The results are shown in Table 2:

TABLE 2

| | | | | | Young's |
|---|---|---|---|---|---|
| | Maximum | Fracture | Tensile | Breaking | modulus of |
| | force | strength | strength | elongation | elasticity |
| Samples | N | KPa | KPa | % | KPa |
| PLGA electrospun | 4.874 | 385.401 | 2235.736 | 12.207 | 167680.221 |
| membrane | | | | | |
| composite scaffold | 10.111 | 666.852 | 4932.027 | 18.092 | 182749.963 |
| material of Embodiment 1 | | | | | |
| composite scaffold | 13.12 | 844.328 | 6399.578 | 20.72 | 268842.696 |
| material of Embodiment 2 | | | | | |
| composite scaffold | 5.77 | 485.465 | 3258.239 | 16.55 | 105181.774 |
| material of Embodiment 3 | | | | | |
| composite scaffold | 7.375 | 689.407 | 3192.468 | 14.783 | 437042.252 |
| material of Embodiment 4 | | | | | |
| composite scaffold | 10.129 | 1090.393 | 8424.110 | 19.135 | 310431.341 |
| material of Embodiment 5 | | | | | |
| composite scaffold | 5.001 | 538.357 | 4841.700 | 15.086 | 209072.405 |
| material of Embodiment 6 | | | | | |
| composite scaffold | 5.462 | 656.906 | 4551.920 | 19.438 | 190560.441 |
| material of Embodiment 7 | | | | | |
| composite scaffold | 3.981 | 547.155 | 5926.771 | 12.687 | 111679.501 |
| material of Embodiment 8 | | | | | |
| composite scaffold | 4.873 | 536.340 | 5957.428 | 11.568 | 144981.253 |
| material of Embodiment 9 | | | | | |
| composite scaffold | 6.553 | 398.122 | 3956.092 | 16.095 | 170848.495 |
| material of Embodiment 10 | | | | | |
| composite scaffold | 2.873 | 430.025 | 3428.527 | 15.455 | 265741.466 |
| material of Embodiment 11 | | | | | |

3. Immersion experiment. Placing the samples in physiological saline and then placing in a constant temperature incubator, measuring the surface area change rate at 10 min, 20 min, 30 min, 2 h and 24 h. The test results are shown in Table 3:

TABLE 3

| Samples | Immersing time | 10 min | 20 min | 30 min | 2 h | 24 h |
|---|---|---|---|---|---|---|
| PLGA electrospun | Area change rate % | 16.2 | 22.6 | 24.4 | 31.3 | 54.7 |
| membrane | | | | | | |
| composite scaffold material | Area change rate % | 5.3 | 7.7 | 8.2 | 12.2 | 18.5 |
| of Embodiment 1 | | | | | | |
| composite scaffold material | Area change rate % | 5.1 | 7.3 | 8.0 | 11.9 | 18.3 |
| of Embodiment 2 | | | | | | |
| composite scaffold material | Area change rate % | 6.6 | 8.1 | 8.8 | 12.7 | 19.2 |
| of Embodiment 3 | | | | | | |
| composite scaffold material | Area change rate % | 5.5 | 6.9 | 8.2 | 11.9 | 17.8 |
| of Embodiment 4 | | | | | | |
| composite scaffold material | Area change rate % | 7.2 | 9.3 | 11.5 | 16.7 | 20.9 |
| of Embodiment 5 | | | | | | |
| composite scaffold material | Area change rate % | 6.4 | 8.4 | 9.2 | 12.1 | 19.6 |
| of Embodiment 6 | | | | | | |
| composite scaffold material | Area change rate % | 4.1 | 6.6 | 7.6 | 9.5 | 14.2 |
| of Embodiment 7 | | | | | | |
| composite scaffold material | Area change rate % | 8.3 | 10.8 | 14.6 | 18.2 | 22.2 |
| of Embodiment 8 | | | | | | |
| composite scaffold material | Area change rate % | 4.4 | 7.0 | 7.6 | 10.3 | 11.7 |
| of Embodiment 9 | | | | | | |
| composite scaffold material | Area change rate % | 5.2 | 7.5 | 8.4 | 10.6 | 12.0 |
| of Embodiment 10 | | | | | | |
| composite scaffold material | Area change rate % | 4.3 | 7.6 | 9.0 | 11.1 | 12.3 |
| of Embodiment 11 | | | | | | |

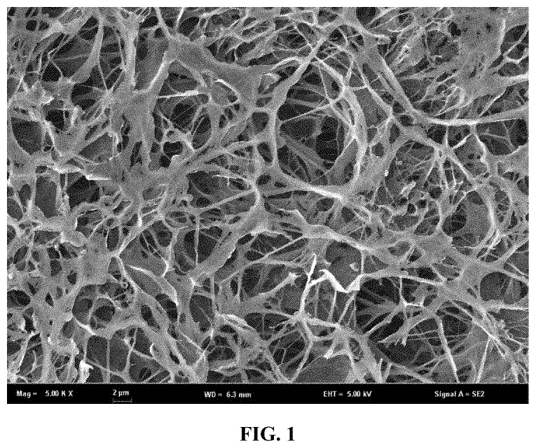

4. Scanning electron microscope test. Technical standard: GB/T 16594-2008. The scanning electron microscope test results of the materials prepared in Embodiment 10 are shown in FIG. 1.

As mentioned above, the present disclosure effectively overcomes various shortcomings in the existing technology and has high industrial utilization value.

The above-mentioned embodiments are just used for exemplarily describing the principle and effects of the present disclosure instead of limiting the present disclosure. Modifications or variations of the above-described embodiments may be made by those skilled in the art without departing from the spirit and scope of the disclosure. Therefore, all equivalent modifications or changes made by those who have common knowledge in the art without departing from the spirit and technical concept disclosed by the present disclosure shall be still covered by the claims of the present disclosure.

1. A composite scaffold material, comprising a charged fiber framework material, wherein the fiber framework material is coated with a positively charged biocompatible material and a negatively charged biocompatible material alternately by means of electrostatic attraction.

2. The composite scaffold material according to claim 1, further comprising one or more of the following:
A1) the forms of the composite scaffold material and/or the fiber framework material are layers and/or spheres and/or tubes;
A2) the fiber framework material is composed of a fiber;
A3) a diameter of the fiber is 3 nm-60000 nm;
A4) a volume percentage of the fiber in the fiber framework material is 20%-90%.

3. The composite scaffold material according to claim 2, further comprising one or more of the following:
B1) the fiber is prepared by electrospinning and/or 3D printing;
B2) the fiber constituting the fiber framework material is charged;
B3) the fiber constituting the fiber framework material is a fiber obtained through charge modification treatment;
B4) the fiber constituting the fiber framework material carries a positively charged group and/or a negatively charged group, wherein the positively charged group is selected from a group consisting of amino group, quaternary ammonium group, and a combination thereof; the negatively charged group is selected from a group consisting of mercapto group, carboxyl group, sulfonic group, phosphate group, and combinations thereof;
B5) the fiber is a biocompatible material, and the fiber is a polymer material;
B6) the fiber is a polymer biocompatible material, and the polymer biocompatible material is an organic synthesized polymer material and/or a natural biopolymer material;
B7) the fiber is doped with a charged doping material, and the doping material is selected from a natural biopolymer material.

4. The composite scaffold material according to claim 3, wherein the natural biopolymer material is selected from a group consisting of polylysine, polyglutamic acid, collagen, silk fibroin, soy protein, elastin, hyaluronic acid, chitosan, carboxymethyl chitosan, carboxymethyl dextran, carboxymethyl glucose, heparin, alginic acid, chondroitin sulfate, carboxymethyl starch and carboxymethyl cellulose; the organic synthesized polymer material is selected from a group consisting of polylactic acid, polyglycolic acid, polycaprolactone, polylactic acid glycolic acid copolymer, polylactic acid caprolactone copolymer, polydioxanone, trimethylene carbonate, polyethylene, polypropylene, polytetrafluoroethylene, polyurethane, polyethylene glycol, polyvinylpyrrolidone, and combinations thereof.

5. The composite scaffold material according to claim 1, further comprising one or more of the following:
C1) the fiber framework material is supplemented with a functional factor and/or a functional polypeptide, an amount of the added functional factor in the fiber framework material is not more than 50 wt %, and an amount of the added functional polypeptide in the fiber framework material is not more than 50 wt %;
C2) the fiber framework material is coated with a positively charged biocompatible material and a negatively charged biocompatible material alternately by means of electrostatic self-assembly.
C3) the positively charged biocompatible material is selected from a group consisting of polylysine, collagen, silk fibroin, fibronectin, laminin, fibrinogen, chitosan, and combinations thereof the negatively charged biocompatible material is selected from a group consisting of polyglutamic acid, collagen, silk fibroin, fibronectin, laminin, fibrinogen, hyaluronic acid, carboxymethyl chitosan, carboxymethyl dextran, carboxymethyl glucose, heparin, alginic acid, chondroitin sulfate, carboxymethyl starch, carboxymethyl cellulose, and combinations thereof;
C4) the positively charged biocompatible material and/or the negatively charged biocompatible material are supplemented with a functional factor and/or a functional polypeptide, an amount of the added functional factor in the positively charged biocompatible material and/or the negatively charged biocompatible material is not more than 50 wt %, and an amount of the added functional polypeptide in the positively charged biocompatible material and/or the negatively charged biocompatible material is not more than 50 wt %.

6. The composite scaffold material according to claim 5, wherein the functional factor is selected from a group consisting of fibronectin, laminin, vascular endothelial growth factor, fibrinogen, nerve growth factor, epidermal growth factor, fibroblast growth factor, transforming growth factor, bone morphogenetic protein, insulin-like growth factor, platelet-derived growth factor, hydroxyapatite, strontium chloride, thrombin, and combinations thereof; the functional polypeptide is selected from a group consisting of RGD polypeptide, polypeptide containing -arginine-glycine-aspartic acid, polypeptide containing -valine-glycine-valine-alanine-proline-glycine, polypeptide containing -isoleucine-lysine-valine-alanine-valine, and combinations thereof.

7. The composite scaffold material according to claim 1, wherein the fiber framework material accounts for 5-95% of a total mass of the composite scaffold material, and the positively charged biocompatible material and the negatively charged biocompatible material account for 5-95% of a total mass of the composite scaffold material.

8. A preparation method for the composite scaffold material according to claim 1, comprising: forming a polymer biocompatible material into a fiber framework material by electrospinning and/or 3D printing, and alternately coating the fiber framework material with a positively charged biocompatible material and a negatively charged biocompatible material by electrostatic self-assembly.

9. The preparation method according to claim 8, further comprising one or more of the following:
D1) an injection speed in electrospinning is 0.001-90 mm/min, a positive voltage of a high voltage generator is 0.1-40 kv, a negative voltage of the high voltage generator is 0.1-10 kv, and a distance between a spinning injector and a receiving device is 3-30 cm, a speed of 3D printing is 0.001 mm-100 mm/min, a printing temperature is −20° C.-400° C., and the electrostatic self-assembly adopts spraying and/or soaking method;
D2) drying and sterilizing are operated after the self-assembly;
D3) electrospinning and/or 3D printing are carried out in the presence of a solvent, and the solvent is selected from a group consisting of formic acid, acetic acid, ethanol, acetone, dimethylformamide, dimethylacetamide, tetrahydrofuran, dimethyl sulfoxide, hexafluoroisopropanol, trifluoroethanol, dichloromethane, trichloromethane, methanol, chloroform, dioxane, trifluoroethane, trifluoroacetic acid, water, normal saline, buffer solution, and combinations thereof.

10. Use of the composite scaffold material according to claim 1 in the preparation of biocompatible materials, preferably in the preparation of tissue adhesive films, tissue repair and regeneration patches, drug carrier materials and/or tissue engineering scaffold materials.
